(19) United States
(12) Patent Application Publication
(10) Pub. No.: US 2024/0000321 A1
(43) Pub. Date: Jan. 4, 2024

(54) Multi-function diagnostic device

(71) Applicant: SANOLLA LTD., Nesher (IL)

(72) Inventors: David Hanuka, Haifa (IL); Doron Adler, Haifa (IL); Noa Bachner-Hinenzon, Zvia (IL); Ezra Salomon, Rakefet (IL); Igor Kagan, Kiryat Bialik (IL)

(21) Appl. No.: 18/251,404

(22) PCT Filed: Oct. 21, 2021
(86) PCT No.: PCT/IB2021/059710

§ 371 Date: May 2, 2023

Related U.S. Application Data
(60) Provisional application No. 63/119,671, filed on Dec. 1, 2020.

(51) Int. Cl. A61B 5/0205; A61B 5/113; A61B 5/332; A61B 5/024; A61B 7/04; A61B 5/00

(52) U.S. Cl. CPC A61B 5/0205; A61B 5/113; A61B 5/332; A61B 5/02416; A61B 7/04; A61B 5/683

(57) ABSTRACT

A medical device (20) includes a case (26) of a size and shape suitable to be held in a hand (24) of a subject (22). An acoustic transducer (36) is disposed in the case and configured to output an acoustical signal in response to acoustic waves that are emitted from a thorax of the subject and received through the front surface of the case when the subject holds the case against the thorax. One or more sensors (46, 48, 50) are disposed on the case and configured to acquire one or more physiological signals from one or more fingers (30) of the subject while the subject holds the case in the hand. Processing circuitry (40) is contained in the case and coupled to receive and process the acoustical signal and the one or more physiological signals and to output data indicative of a medical condition of the subject.

### CROSS-REFERENCE TO RELATED APPLICATION

This application claims the benefit of U.S. Provisional Patent Application 63/119,671, filed Dec. 1, 2020, which is incorporated herein by reference.

### FIELD OF THE INVENTION

The present invention relates generally to devices and methods for medical diagnosis, and particularly to diagnosis of respiratory and cardiac conditions.

### BACKGROUND

Auscultation has been a key technique in medical diagnosis for centuries. In auscultation, the medical practitioner listens to the internal sounds of the body, typically using a stethoscope. Auscultation is most commonly performed for the purpose of examining the circulatory and respiratory systems, and thus diagnosing conditions of the heart and lungs in particular. In more recent years, electronic stethoscopes and methods of digital processing of body sounds have become available, in order to enhance and supplement the practitioner's auditory capabilities.

PCT International Publication WO 2017/141165, whose disclosure is incorporated herein by reference, describes apparatus for detecting sound waves emanating from a body of a subject. The apparatus includes a housing and a membrane, disposed at an opening of the housing. The membrane is configured to deflect, when an outer face of the membrane contacts the body, responsively to the sound waves impinging on the membrane. The apparatus further includes a piezoelectric microphone, disposed within the housing, configured to detect vibrations of air caused by the deflection of the membrane, and to generate a microphone output in response thereto. An accelerometer, disposed on an inner face of the membrane, deflects, along with the membrane, at frequencies below a minimum frequency that is detectable by the piezoelectric microphone, and generate an accelerometer output in response thereto. A processor processes the microphone output and the accelerometer output, and generates, responsively to the processing, a sound signal that represents the impinging sound waves.

As another example, PCT International Publication WO 2019/048960, whose disclosure is incorporated herein by reference, describes a medical device, which includes a case having a front surface that is configured to be brought into contact with a body of a living subject. A microphone is contained in the case and configured to sense acoustic waves emitted from the body and to output an acoustic signal in response thereto. A proximity sensor is configured to output a proximity signal indicative of contact between the front surface and the body. At least one speaker is configured to output audible sounds. Processing circuitry is coupled to detect, in response to the proximity signal, that the front surface is in contact with the body, and in response to the detected contact, to process the acoustic signal so as to generate an audio output and to convey the audio output to the at least one speaker.

PCT International Publication WO 2019/048961, whose disclosure is incorporated herein by reference, describes diagnosis of pathologies using infrasonic signatures. In one embodiment, a medical device includes an acoustic transducer, which is configured to sense infrasonic waves emitted from a body of a living subject with a periodicity determined by a periodic physiological activity and to output an electrical signal in response to the sensed waves. At least one speaker is configured to output audible sounds in response to an electrical input. Processing circuitry is configured to process the electrical signal so as to generate a frequency-stretched signal in which infrasonic frequency components of the electrical input are shifted to audible frequencies while preserving the periodicity of the periodic physiological activity in the frequency-stretched signal, and to input the frequency-stretched signal to the at least one speaker.

### SUMMARY

Embodiments of the present invention that are described hereinbelow provide improved medical diagnostic devices and methods for their use.

There is therefore provided, in accordance with an embodiment of the invention, a medical device, including a case of a size and shape suitable to be held in a hand of a subject, the case having front and rear surfaces. An acoustic transducer is disposed in the case and configured to output an acoustical signal in response to acoustic waves that are emitted from a thorax of the subject and received through the front surface of the case when the subject holds the front surface of the case against the thorax. One or more sensors are disposed on the case and configured to acquire one or more physiological signals from one or more fingers of the subject while the subject holds the case in the hand. Processing circuitry is contained in the case and coupled to receive and process the acoustical signal and the one or more physiological signals and to output data indicative of a medical condition of the subject.

In some embodiments, the case includes a receptacle, which is fixed to the rear surface of the case and is shaped and oriented to receive one of the fingers of the subject and which contains a sensor for acquiring at least one of the physiological signals from the one of the fingers. In a disclosed embodiment, the sensor includes one or more optical emitters, which are configured to direct optical radiation toward the one of the fingers in the receptacle, and an optical receiver, which is configured to output the physiological signal in response to the optical radiation that is received from the one of the fingers, wherein the physiological signal is indicative of an oxygen saturation of blood in the one of the fingers.

Additionally or alternatively, the one or more sensors include an electrode, which is disposed on the case and is configured to contact one of the fingers of the subject, and wherein the processing circuitry is configured to extract an electrocardiogram (ECG) from the physiological signal acquired by the electrode. In a disclosed embodiment, the device includes a further electrode disposed on the front surface of the case and configured to contact the thorax of the subject, wherein the processing circuitry is configured to measure the ECG between the electrode contacting the one of the fingers and the further electrode contacting the thorax.

Further additionally or alternatively, the processing circuitry is configured to extract from the acoustical signal, a seismocardiogram (SCG) of the subject, to make a comparison between respective features of the SCG and the ECG, and to output the data responsively to the comparison. In one embodiment, the one or more sensors include one or more optical emitters, which are configured to direct optical radiation toward a finger of the subject, and an optical receiver, which is configured to output a further physiological signal in response to the optical radiation that is received from the finger, and the processing circuitry is configured to extract a pulse waveform from the further physiological signal and to compare the pulse waveform to at least one of the ECG and the SCG.

In some embodiments, the front surface of the case includes a membrane, which vibrates in response to the acoustic waves, and the acoustic transducer is coupled to sense a vibration of the membrane. In a disclosed embodiment, the acoustic transducer that is coupled to sense the vibration of the membrane is a first acoustic transducer and is configured to output a first acoustical signal in response to the vibration of the membrane, and the device includes a second acoustic transducer, which is configured to output a second acoustical signal in response to ambient acoustic waves that are incident on the case, and the processing circuitry is configured to generate a measure of a physiological activity in the thorax responsively to a difference between the first and second acoustical signals. In one embodiment, the device includes a user interface, which is configured to prompt the subject to vocalize one or more predefined sounds, wherein the processing circuitry is configured to process the acoustical signal received from both the first and second acoustic transducers while the subject vocalizes the one or more predefined sounds.

In another embodiment, the device includes a pressure sensor, which is configured to sense a force applied between the front surface of the case and the thorax, wherein the processing circuitry is configured to output an instruction to the subject to modify the applied force responsively to the sensed force.

There is also provided, in accordance with an embodiment of the invention, medical apparatus, including at least one electrode, which is configured to acquire an electrical signal from a body surface of a subject. An acoustic transducer is configured to output an acoustical signal in response to acoustic waves that are emitted from a thorax of the subject in synchronization with the electrical signal. A processor is configured to process the acoustical signal in order to extract a seismocardiogram (SCG) of the subject, to process the electrical signal in order to extract an electrocardiogram (ECG) of the subject, to make a comparison between respective features of the SCG and the ECG, and to output data indicative of a medical condition of the subject responsively to the comparison.

In a disclosed embodiment, the processor is configured identify a periodic feature in the ECG and to segment the SCG using the identified periodic feature and the synchronization of the acoustical signal with the electrical signal.

There is additionally provided, in accordance with an embodiment of the invention, an electronic stethoscope, including a head, which is configured to be held, by a practitioner, in contact with a thorax of a subject, and which includes a first acoustic transducer, which is configured to output a first acoustical signal in response to acoustic waves that are emitted from the thorax, and a second acoustic transducer, which is configured to output a second acoustical signal in response to ambient acoustic waves that are incident on the head. At least one eartip is configured to output sounds representing the acoustic waves to an ear of the practitioner, and includes a third acoustic transducer, which is configured to output a third acoustical signal in response to the sounds output by the at least one eartip. Processing circuitry is coupled to process the first acoustical signals so as to generate the sounds for output by the at least one eartip while filtering out ambient noise and distortion responsively to the second and third acoustical signal.

In a disclosed embodiment, the first acoustical signal includes infrasonic components, and the processing circuitry is configured to convert the infrasonic components to audible frequencies and to incorporate the converted infrasonic components in the sounds for output by the at least one eartip.

There is further provided, in accordance with an embodiment of the invention, a method for sensing, which includes providing a case of a size and shape suitable to be held in a hand of a subject. The case contains an acoustic transducer configured to output an acoustical signal in response to acoustic waves that are emitted from a thorax of the subject and received through a front surface of the case when the subject holds the front surface of the case against the thorax. One or more sensors are disposed on the case and configured to acquire one or more physiological signals from one or more fingers of the subject while the subject holds the case in the hand. While the subject holds the case in contact with the thorax, the acoustical signal and the one or more physiological signals are received and processed so as to output data indicative of a medical condition of the subject.

There is moreover provided, in accordance with an embodiment of the invention, a method for sensing, which includes acquiring an electrical signal from a body surface of the subject and acquiring an acoustical signal in response to acoustic waves that are emitted from a thorax of the subject in synchronization with the electrical signal. The acoustical signal is processed in order to extract a seismocardiogram (SCG) of the subject, and the electrical signal is processed in order to extract an electrocardiogram (ECG) of the subject. A comparison is made between respective features of the SCG and the ECG, and data indicative of a medical condition of the subject are output responsively to the comparison.

The present invention will be more fully understood from the following detailed description of the embodiments thereof, taken together with the drawings in which:

### BRIEF DESCRIPTION OF THE DRAWINGS

FIG. 1A is a schematic pictorial illustration showing a medical diagnostic device in use by a subject, in accordance with an embodiment of the invention;

FIG. 1B is a schematic detail view of the device of FIG. 1A;

FIG. 2 is a schematic sectional view showing functional components of the medical diagnostic device of FIGS. 1A/B, in accordance with an embodiment of the invention;

FIG. 3 is a flow chart that schematically illustrates a method for medical diagnosis, in accordance with an embodiment of the invention;

FIG. 4A is a schematic pictorial view of an electronic stethoscope, in accordance with an embodiment of the invention; and

FIG. 4B is a block diagram showing functional components of the electronic stethoscope of FIG. 4A, in accordance with an embodiment of the invention.

### DETAILED DESCRIPTION OF EMBODIMENTS

### Overview

Auscultation can provide a wealth of diagnostic information, for example as described in the PCT international publications cited above. Generally speaking, however, only skilled medical practitioners are capable of using stethoscopes properly and deriving diagnostic information from the chest sounds that they provide. To make a diagnosis, medical practitioners typically use the stethoscope in a series of tests together with other instruments, such as an electrocardiogram (ECG), pulse oximeter, and thermometer. In many cases, the patient's condition can change rapidly, requiring frequent follow-up testing. There is thus a need for devices and methods that can be used in auscultation-based screening and follow-up without the involvement of a skilled clinician.

Some embodiments of the present invention address this need by providing integrated medical devices that enable rapid, reliable diagnostic follow-up by the patient or a caregiver, without special training or medical skills. The devices can be used either in the clinic or in the patient's home, and have built-in communication capabilities that can provide the diagnostic information they collect to a medical professional and/or to a remote computer for analysis.

The medical device in these embodiments comprises a case of a size and shape suitable to be held in the hand of a subject. The subject holds the front surface of the case against his or her chest. An acoustic transducer in the case senses acoustic waves that are emitted from the subject's thorax and received through the front surface. In addition, one or more sensors disposed on the case acquire one or more continuous physiological signals from the finger, while the subject holds the case against his or her chest. Processing circuitry contained in the case receives and processes both the acoustical signal from the acoustic transducer and the physiological signal acquired by the sensor, and on this basis outputs data indicative of a medical condition of the subject.

The terms “acoustical signal” and “physiological signal” are used in the present description and in the claims, for the sake of convenience and clarity, to refer to the electrical signals that are output respectively by the acoustic transducer and by other sensors in the device. The data output by the processing circuitry in response to these signals may simply comprise digitized versions of the signals (typically following some filtering and amplification), which are output to another computer, for example, for further processing and display. Alternatively or additionally, the processing circuitry in the case of the device may perform further data analysis and may then output diagnostic values that are indicative of the subject's condition. Techniques for this sort of data analysis are described hereinbelow, as well as in the PCT international publications cited above.

In embodiments of the present invention, the device may contain various types of sensors. In one embodiment, a receptacle is fixed to the rear surface of the case and is shaped and oriented to receive one of the fingers of the subject. The receptacle contains one or more optical emitters, which direct optical radiation toward the finger in the receptacle, and an optical receiver, which outputs the physiological signal in response to the optical radiation that is received from the finger. In this case, the sensor serves as a pulse oximeter, and the physiological signal is indicative of the oxygen saturation of the blood in the finger, as well as providing a pulse waveform indicative of blood flow in the finger. Changes in the blood oxygen saturation can be used together with the sounds received by the acoustic transducer as an indication of the severity of the subject's respiratory or cardiac condition.

Additionally or alternatively, the device comprises an electrode, which is disposed on the case in a location chosen so as to contact one of the fingers of the subject while the subject holds the case. The electrode thus senses electrical activity in the subject' body. The processing circuitry can then extract an electrocardiogram (ECG) from the physiological signal acquired by the electrode. A further electrode may be disposed on the front surface of the case so as to contact the subject's thorax, thus enabling the processing circuitry to measure the ECG between the finger and the thorax.

In some embodiments, processing circuitry within or outside the case extracts a seismocardiogram (SCG) from the acoustical signal provided by the acoustic transducer. As the ECG and SCG are acquired simultaneously, in synchronization, the processing circuitry is able to make a comparison between respective features of the SCG and the ECG, and to output diagnostic data based the comparison. The pulse waveform provided by the optical sensor can be used in this analysis, as well. These synchronized, comparative analyses may be performed advantageously on the basis of the data provided by the sort of handheld device that is described herein. Alternatively, such analyses may be carried out using other sorts of sensing systems in which the SCG and ECG are suitably synchronized.

Other embodiments described below provide electronic stethoscopes, typically for use by medical practitioners. These stethoscopes may offer sensing and processing capabilities similar to those of the handheld devices described above, with enhancements suitable for professional use. In contrast to the handheld device, however, such electronic stethoscopes output sounds representing acoustic waves received from the patient's thorax to the practitioner's ears. In one embodiment, the electronic stethoscope includes multiple acoustic transducers, along with processing circuitry that uses the acoustical signals output by the transducers in filtering out ambient noise and distortion from the sounds that it outputs.

### Handheld Diagnostic Device

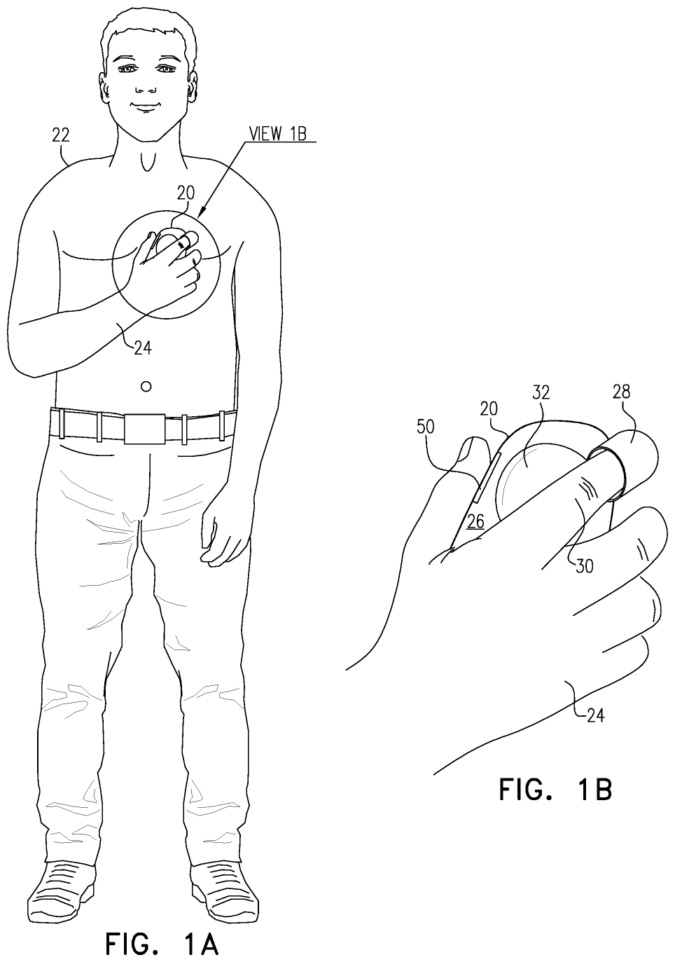

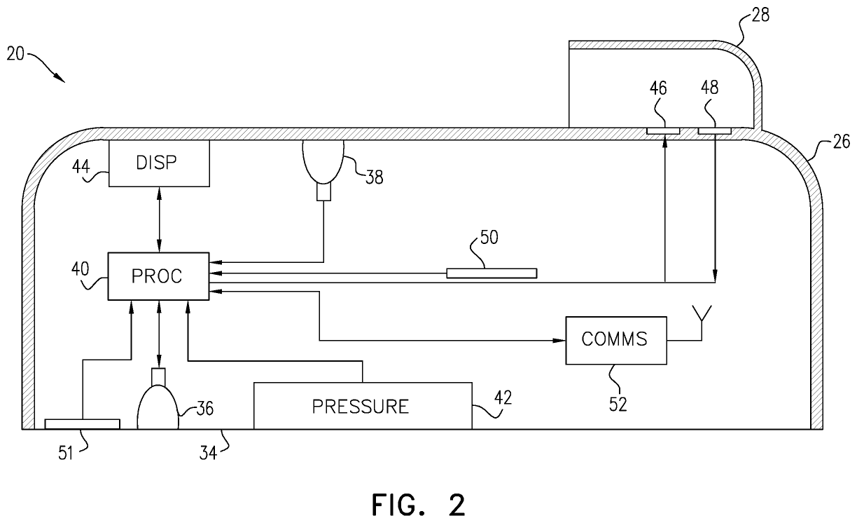

Reference is now made to FIGS. 1A, 1B and 2, which schematically show a medical diagnostic device 20, in accordance with an embodiment of the invention. FIG. 1A is a pictorial illustration showing device 20 in use by a human subject 22, while FIG. 1B is a detail view of the device of FIG. 1A. FIG. 2 is a sectional view showing functional components of device of 20.

Device 20 comprises a case 26 of a size and shape suitable to be held in a hand 24 of subject 22, who presses case 26 against his chest. The front surface of case 26 comprises a membrane 34, which vibrates in response to acoustic waves emitted from the subject's thorax, for example due to cardiac and respiratory activity. An acoustic transducer 36 senses vibrations of the membrane. Transducer 36 typically comprises a microphone, which converts the vibrations of membrane 34 into electrical signals. Additionally or alternatively, transducer 36 may comprise an accelerometer, which is useful in detecting infrasonic vibrations, i.e., vibrations at frequencies below 20 Hz (which is considered to be the lower limit of the audible range). The use of an accelerometer for this purpose is described, for example, in the above-mentioned PCT International Publication WO 2017/141165.

A receptacle 28 is fixed to the rear surface of case 26 and is shaped and oriented to receive the tip of a finger 30 of subject 22 while the subject holds the case in his hand 24. In the pictured embodiment, receptacle 28 has the form of a sort of thimble. Alternatively, the receptacle may be configured differently, for example as a clip on the rear surface of the case. One or more sensors in receptacle 28 acquire physiological signals from finger 30. In the pictured example, these sensors include a pulse oximetry sensor, comprising an optical emitter 46 and an optical receiver 48.

Other sensors may also be disposed on or in case 26. In the present embodiment, an electrode 50 on the case contacts one of the fingers (for example, the subject's thumb) and serves as an ECG sensor. For purposes of ECG sensing, an additional electrode 51 is positioned on the front surface of the case and contacts the skin of subject 22 while device 20 is in use. Electrode 51 thus functions as the ECG common electrode, and the ECG signal is measured between electrode 50 and electrode 51. Alternatively, an additional ECG electrode (not shown) may be applied to the subject's body.

Additionally or alternatively, device 20 may comprise other sorts of physiological sensors, such as a temperature sensor.

For purposes of pulse oximetry, optical emitter 46 typically comprises one or more light-emitting diodes (LEDs), which emit visible radiation, such as red light at a wavelength of 660 nm, and infrared radiation, for example at 940 nm. Optical receiver 48 typically comprises a suitable photodiode. Although the sensor is shown in FIG. 2 to be operating in a reflective mode, with the emitter and receiver side-by-side, the emitter and receiver may alternatively be positioned on opposite sides of receptacle, so that the pulse oximeter operates in a transmissive mode.

Processing circuitry 40 within case 26 receives and processes the signals from the transducers and sensors in device 20. Processing circuitry 40 includes analog front-end circuits, which amplify and filter the signals, and an analog/digital converter, which converts the signals to digital form. Processing circuitry 40 typically applies digital signal processing to the digitized signals for purposes of noise reduction and background subtraction. To perform these functions, processing circuitry 40 may comprise dedicated or programmable digital logic circuits. Additionally or alternatively, processing circuitry 40 comprises a microprocessor or an embedded microcontroller, which is programmed in software or firmware to carry out at least some of the digital processing and control functions that are described herein. In this latter case, processing circuitry 40 may also analyze the signals that it receives from the sensors in device 20 in order to generate and output diagnostic indicators with respect to subject 22. These sorts of processing functions are described, for example, in the above-mentioned PCT International Publication WO 2019/048961.

Processing circuitry 40 outputs digital data via a user interface 32 of device 20 and/or via a communications interface 52. User interface 32, for example, may comprise a touch-sensitive display screen, which is driven by a display driver 44 to present information and receive inputs from a user, such as subject 22. Additionally or alternatively, the user interface of device 20 may comprise a voice interface. Communications interface 52 typically comprises a wireless transceiver, for example a Bluetooth® transceiver for communicating with a nearby computing device, such as a local computer or smartphone. Alternatively or additionally, communications interface 52 comprises a Wi-Fi or cellular interface for communicating with a remote computing device, such as a server. The computing device that receives the digital data from processing circuitry 40 via communications interface 52 may perform further diagnostic processing, comparative analyses, and data storage, for example.

As shown in FIG. 2, in addition to acoustic transducer 36, which senses the vibration of membrane 34, device 20 comprises a second acoustic transducer 38, which senses ambient acoustic waves that are incident on case 26. After digitizing the acoustical signals output by transducers 36 and 38, processing circuitry 40 takes a difference between the signals in order to cancel background noise from the thoracic vibrations sensed by transducer 36 and thus to provide a more precise measure of a physiological activity in the thorax. For purposes of cardiac diagnosis, the measure may take the form of a seismocardiogram, as described below. Additionally or alternatively, for respiratory diagnosis, processing circuitry 40 may compute a spectral distribution of the vibrational energy over successive respiratory cycles, for example an infrasonic “signature” as described in the above-mentioned PCT International Publication WO 2019/048961.

In some embodiments, processing circuitry 40 outputs instructions and prompts to subject 22 via user interface 32. These instructions may guide the subject in improving the quality of the measured signal. For this purpose, in the embodiment shown in FIG. 2, device 20 comprises a pressure sensor 42, which senses the force applied between the front surface of case 26 and the subject's thorax. Processing circuitry 40 checks whether the measured force is within a desired target range and, if not, outputs an instruction to subject 22 to increase or decrease the applied force in order to bring it into range.

In another embodiment, processing circuitry 40 prompts subject 22 to vocalize one or more predefined sounds while the subject holds device 20 against his chest. Processing circuitry 40 processes the acoustical signals output by transducer 36 due to the vibration induced in membrane 34 while the subject vocalizes the sounds. The signals in this case can provide additional useful diagnostic information with respect to cardiac and respiratory conditions, such as accumulation of fluid in the subject's chest. Acoustic transducer 38 may sense the actual sounds output from the subject's mouth, thus providing additional data for comparative analysis.

### Seismocardiogram Analysis

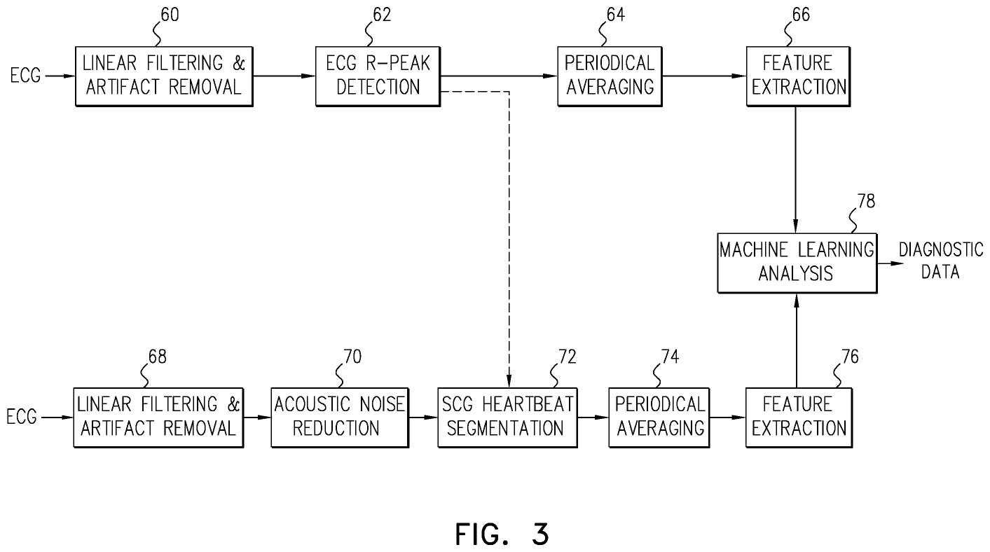

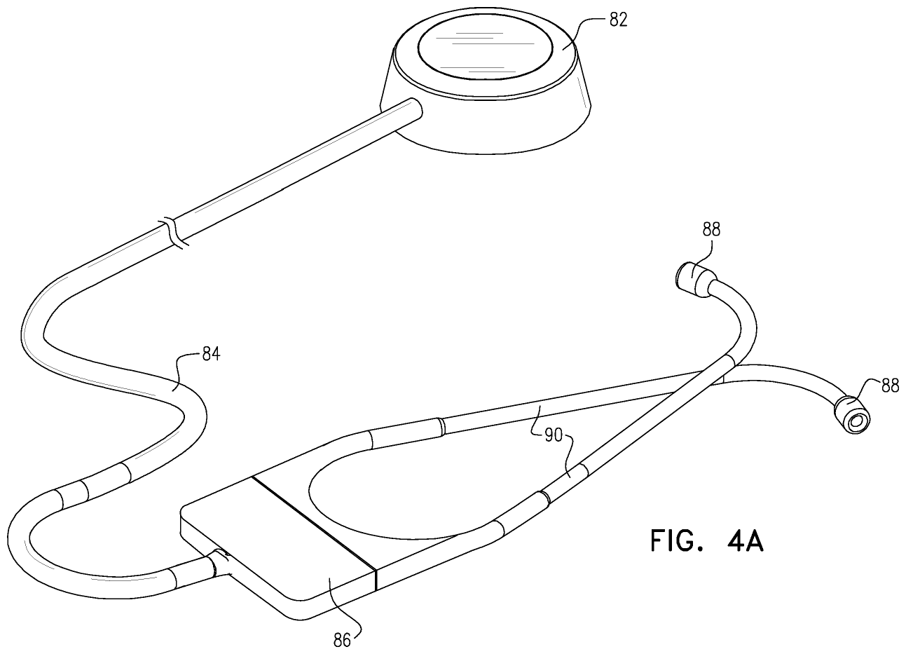

FIG. 3 is a flow chart that schematically illustrates a method for medical diagnosis using ECG and seismocardiogram (SCG) signals, in accordance with an embodiment of the invention. The method is described here, for the sake of concreteness and clarity, with reference to device 20. Alternatively, this method may be implemented using other equipment with suitable electrical and acoustic sensing capabilities.

Processing circuitry 40 receives an ECG input, for example from electrode 50, and digitizes and filters the signal to remove artifacts and noise, at an ECG filtering step 60. The processing circuitry applies a peak detection algorithm to detect the periodic R-wave in the ECG signal, at a peak detection step 62. Using the R-wave peak as an annotation point, processing circuitry 40 averages the ECG signals over multiple heart cycles (with adjustment as needed for heart rate variations), at an ECG averaging step 64. The processing circuitry then extracts features of interest from the averaged ECG signal, such as the relative timing, amplitudes, and shapes of the characteristic parts of the ECG signal (such as the P, Q, S and T waves), at an ECG feature extraction step 66.

Processing circuitry 40 receives an acoustical input, for example from transducers 36 and 38, and digitizes and filters the acoustical signals to remove artifacts and noise, at an SCG filtering step 68. The processing circuitry subtracts the background signal output by transducer 38 from the chest vibration signal output by transducer 36, at an acoustic noise reduction step 70. Based on the subtracted signal, processing circuitry 40 computes the SCG, representing the total amplitude of the vibrations as a function of time within a frequency range of interest, for example frequencies up to 100 Hz.

Processing circuitry 40 uses the R-wave peak output detected in each heart cycle at step 62 as an annotation point for segmenting the SCG into successive heart cycles, at an SCG segmentation step 72. The processing circuitry aligns the segmented SCG signals over multiple heart cycles using this annotation and averages the aligned signals, at an SCG averaging step 74. Processing circuitry 40 extracts features of interest from the averaged SCG signal, at an SCG feature extraction step 76. The features of interest in this case may include, for example, peaks in the SCG that are associated with opening and closing of the valves in the heart, such as the atrial and mitral valves. The timing of these peaks, relative to the ECG R-wave and to one another, and the amplitudes of the peaks can indicate problems in the electrical and mechanical functioning of the heart.

Processing circuitry 40 analyzes the features of the ECG and SCG that were extracted at steps 66 and 76 in order to generate diagnostic data with respect to the heart of subject 22, at an analysis step 78. Alternatively or additionally, this analysis may be performed by an external computer (not shown), which receives the extracted features via communications interface 52 of device 20. Machine learning techniques can be useful in this analysis. For example, synchronized ECG and SCG signals may be collected from many patients with known diagnoses, and the features of these signals may be used in training a neural network or other suitable classifier to associate such features with diagnoses. Features of interest for this purpose include the locations and widths of the peaks in the SCG, delay between the ECG and SCG peaks, and any murmurs in the SCG. Following this training, the neural network may be used at step 78 to generate diagnostic data for patients whose diagnosis is not yet known.

In addition to the ECG and SCG signals, processing circuitry 40 may also extract a peripheral pulse waveform from the signal output by optical receiver 48 in device 20, or from a conventional pulse oximeter attached to the subject's finger. In one embodiment, processing circuitry 40 compares this peripheral pulse waveform to the ECG and/or the SCG as a further diagnostic indicator. For example, the delay of the pulse waveform in finger 30 relative to the ejection of blood from the heart as seen in the SCG can be indicative of circulatory problems.

### Electronic Stethoscope with Improved Audio Quality

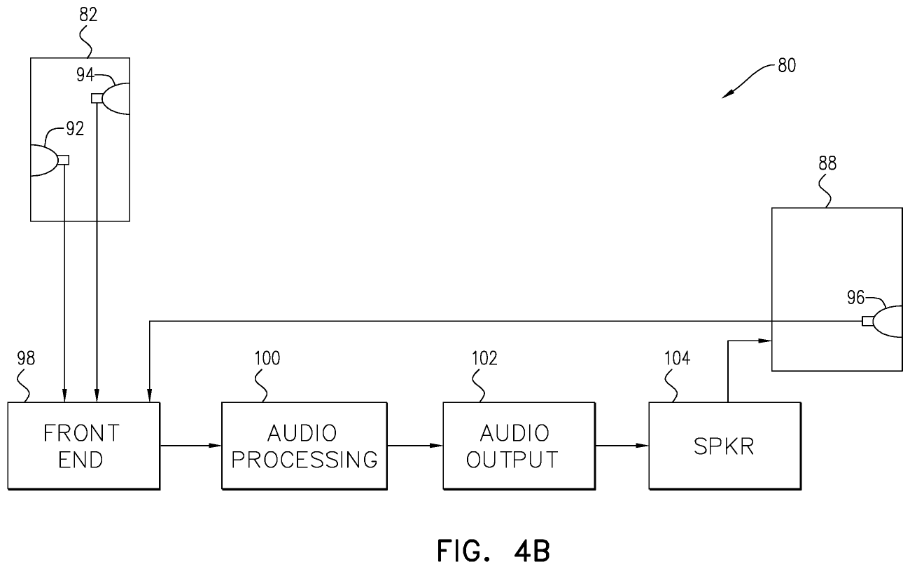

Reference is now made to FIGS. 4A and 4B, which schematically illustrate an electronic stethoscope 80, in accordance with an embodiment of the invention. FIG. 4A is a schematic pictorial view of the electronic stethoscope, while FIG. 4B is a block diagram showing functional components of the electronic stethoscope. In contrast to the embodiment shown in FIG. 1A, electronic stethoscope 80 is designed for use by a medical practitioner, with parts shaped to resemble those of a conventional acoustic stethoscope.

Electronic stethoscope 80 comprises a head 82, which is held, by a practitioner, in contact with the thorax of a subject. As in device 20 (FIG. 2), head 82 comprises two acoustic transducer 92 and 94. Transducer 92 is mounted on the face of head 82 that contacts the subject's thorax and thus outputs an acoustical signal in response to acoustic waves that are emitted from the thorax. Transducer 94 is mounted in head 82 away from this face and outputs an acoustical signal in response to ambient acoustic waves that are incident on the head. At least transducer 92 may be capable of sensing infrasonic waves, in additional to acoustic waves in the audible range.

Head 82 is connected by a cable 84 to a processing unit 86, which generates sounds for transmission via acoustic tubes 90 to a pair of eartips 88. (Alternatively, a single eartip may be used.) Eartips 88 output sounds representing the acoustic waves received from the subject's thorax to the practitioner's ears. In addition, at least one of eartips 88 contains an additional acoustic transducer 96, which outputs an acoustical signal in response to the sounds that are output from the eartip. By detecting the actual sounds that are output to the practitioner's ears, transducer 96 provides an indication of distortion and variations that may be introduced at various points in the audio train of electronic stethoscope 80, including both electronic components and acoustic components of the audio train.

The processing circuitry in electronic stethoscope 80, as illustrated in FIG. 4B, may be distributed between processing unit 86 and head 82, or it may be contained entirely within the processing unit. The processing circuitry includes front-end circuits 98, which receive, amplify, and digitize the acoustical signals output by transducers 92, 94 and 96. An audio processing circuit 100 filters the digital signals and specifically uses the signals from transducers 94 and 96 to remove ambient noise and distortion from the signal output by transducer 92. The resulting filtered digital audio signal is converted back to analog form and amplified by an audio output circuit 102, which feeds the output signal to a speaker 104. The audio output of speaker 104 passes through acoustic tubes 90 to eartips 88.

In one embodiment, the ambient noise detected by transducer 94 is used in reducing orthogonal components correlated with the ambient noise signal from the acoustic data detected by transducer 92 using dynamic linear filter (whose coefficients are adjusted according to the signals output by transducer 94). The processing circuitry analyzes the spectral response and the intensity of the signal output by transducer 96 to verify that the analog signal at the end of the processing chain, which includes the output of speaker 104 and acoustic tubes 90, retains the desired spectral response and intensity (to protect the user's ears and ensure good audio quality).

As noted earlier, the acoustical signal output by transducer 92 may include infrasonic components, which contain important diagnostic information. To make this information audible to the medical practitioner, audio processing circuit 100 converts the infrasonic components to audible frequencies and incorporates the converted infrasonic components into the output sounds that are generated by audio output circuit 102. Processing techniques that can be used for this purpose are described, for example, in the above-mentioned PCT International Publication WO 2019/048961.

It will be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

1. A medical device, comprising:
a case of a size and shape suitable to be held in a hand of a subject, the case having front and rear surfaces;
an acoustic transducer disposed in the case and configured to output an acoustical signal in response to acoustic waves that are emitted from a thorax of the subject and received through the front surface of the case when the subject holds the front surface of the case against the thorax;
one or more sensors disposed on the case and configured to acquire one or more physiological signals from one or more fingers of the subject while the subject holds the case in the hand; and
processing circuitry contained in the case and coupled to receive and process the acoustical signal and the one or more physiological signals and to output data indicative of a medical condition of the subject.

2. The device according to claim 1, wherein the case comprises a receptacle, which is fixed to the rear surface of the case and is shaped and oriented to receive one of the fingers of the subject and which contains a sensor for acquiring at least one of the physiological signals from the one of the fingers.

3. The device according to claim 2, wherein the one or more sensors comprise one or more optical emitters, which are configured to direct optical radiation toward the one of the fingers in the receptacle, and an optical receiver, which is configured to output the physiological signal in response to the optical radiation that is received from the one of the fingers, wherein the physiological signal is indicative of an oxygen saturation of blood in the one of the fingers.

4. The device according to claim 1, wherein the one or more sensors comprise an electrode, which is disposed on the case and is configured to contact one of the fingers of the subject, and wherein the processing circuitry is configured to extract an electrocardiogram (ECG) from the physiological signal acquired by the electrode.

5. The device according to claim 4, and comprising a further electrode disposed on the front surface of the case and configured to contact the thorax of the subject, wherein the processing circuitry is configured to measure the ECG between the electrode contacting the one of the fingers and the further electrode contacting the thorax.

6. The device according to claim 4, wherein the processing circuitry is configured to extract from the acoustical signal, a seismocardiogram (SCG) of the subject, to make a comparison between respective features of the SCG and the ECG, and to output the data responsively to the comparison.

7. The device according claim 4, wherein the one or more sensors comprise one or more optical emitters, which are configured to direct optical radiation toward a finger of the subject, and an optical receiver, which is configured to output a further physiological signal in response to the optical radiation that is received from the finger, and wherein the processing circuitry is configured to extract a pulse waveform from the further physiological signal and to compare the pulse waveform to at least one of the ECG and the SCG.

8. The device according to claim 1, wherein the front surface of the case comprises a membrane, which vibrates in response to the acoustic waves, and wherein the acoustic transducer is coupled to sense a vibration of the membrane.

9. The device according to claim 8, wherein the acoustic transducer that is coupled to sense the vibration of the membrane is a first acoustic transducer and is configured to output a first acoustical signal in response to the vibration of the membrane, and
wherein the device comprises a second acoustic transducer, which is configured to output a second acoustical signal in response to ambient acoustic waves that are incident on the case, and
wherein the processing circuitry is configured to generate a measure of a physiological activity in the thorax responsively to a difference between the first and second acoustical signals.

10. The device according to claim 9, and comprising a user interface, which is configured to prompt the subject to vocalize one or more predefined sounds, wherein the processing circuitry is configured to process the acoustical signal received from both the first and second acoustic transducers while the subject vocalizes the one or more predefined sounds.

11. The device according to claim 1, and comprising a pressure sensor, which is configured to sense a force applied between the front surface of the case and the thorax, wherein the processing circuitry is configured to output an instruction to the subject to modify the applied force responsively to the sensed force.

12. Medical apparatus, comprising:
at least one electrode, which is configured to acquire an electrical signal from a body surface of a subject;
an acoustic transducer, which is configured to output an acoustical signal in response to acoustic waves that are emitted from a thorax of the subject in synchronization with the electrical signal; and
a processor, which is configured to process the acoustical signal in order to extract a seismocardiogram (SCG) of the subject, to process the electrical signal in order to extract an electrocardiogram (ECG) of the subject, to make a comparison between respective features of the SCG and the ECG, and to output data indicative of a medical condition of the subject responsively to the comparison.

13. The apparatus according to claim 12, wherein the processor is configured to identify a periodic feature in the ECG and to segment the SCG using the identified periodic feature and the synchronization of the acoustical signal with the electrical signal.

14. An electronic stethoscope, comprising:
a head, which is configured to be held, by a practitioner, in contact with a thorax of a subject, and which comprises:
a first acoustic transducer, which is configured to output a first acoustical signal in response to acoustic waves that are emitted from the thorax; and
a second acoustic transducer, which is configured to output a second acoustical signal in response to ambient acoustic waves that are incident on the head;
at least one eartip, which is configured to output sounds representing the acoustic waves to an ear of the practitioner, and which comprises a third acoustic transducer, which is configured to output a third acoustical signal in response to the sounds output by the at least one eartip; and
processing circuitry, which is coupled to process the first acoustical signals so as to generate the sounds for output by the at least one eartip while filtering out ambient noise and distortion responsively to the second and third acoustical signal.

15. The electronic stethoscope according to claim 14, wherein the first acoustical signal includes infrasonic components, and wherein the processing circuitry is configured to convert the infrasonic components to audible frequencies and to incorporate the converted infrasonic components in the sounds for output by the at least one eartip.

16. A method for sensing, comprising:
providing a case of a size and shape suitable to be held in a hand of a subject, the case containing an acoustic transducer configured to output an acoustical signal in response to acoustic waves that are emitted from a thorax of the subject and received through a front surface of the case when the subject holds the front surface of the case against the thorax, and one or more sensors disposed on the case and configured to acquire one or more physiological signals from one or more fingers of the subject while the subject holds the case in the hand; and
while the subject holds the case in contact with the thorax, receiving and processing the acoustical signal and the one or more physiological signals so as to output data indicative of a medical condition of the subject.

17. The method according to claim 16, wherein the case comprises a receptacle, which is fixed to the rear surface of the case and is shaped and oriented to receive one of the fingers of the subject and which contains a sensor for acquiring at least one of the physiological signals from the one of the fingers.

18. The method according to claim 17, wherein the one or more sensors comprise one or more optical emitters, which are configured to direct optical radiation toward the one of the fingers in the receptacle, and an optical receiver, which is configured to output the physiological signal in response to the optical radiation that is received from the one of the fingers, wherein the physiological signal is indicative of an oxygen saturation of blood in the one of the fingers.

19. The method according to claim 16, wherein the one or more sensors comprises an electrode, which is disposed on the case and is configured to contact the one of the fingers of the subject, and wherein processing the physiological signal comprises extracting an electrocardiogram (ECG) from the physiological signal acquired by the electrode.

20. The method according to claim 19, wherein a further electrode is disposed on the front surface of the case and configured to contact the thorax of the subject, and wherein extracting the ECG comprises measuring the ECG between the electrode contacting the one of the fingers and the further electrode contacting the thorax.

21. The method according to claim 19, wherein processing the acoustical signal comprises extracting from the acoustical signal a seismocardiogram (SCG) of the subject, making a comparison between respective features of the SCG and the ECG, and outputting the data responsively to the comparison.

22. The method according claim 21, wherein the one or more sensors comprise one or more optical emitters, which are configured to direct optical radiation toward a finger of the subject, and an optical receiver, which is configured to output a further physiological signal in response to the optical radiation that is received from the finger, and wherein processing the physiological signal comprises extracting a pulse waveform from the further physiological signal and comparing the pulse waveform to at least one of the ECG and the SCG.

23. The method according to claim 16, wherein the front surface of the case comprises a membrane, which vibrates in response to the acoustic waves, and wherein the acoustic transducer is coupled to sense a vibration of the membrane.

24. The method according to claim 23, wherein the acoustic transducer that is coupled to sense the vibration of the membrane is a first acoustic transducer and is configured to output a first acoustical signal in response to the vibration of the membrane, and wherein the case contains a second acoustic transducer, which is configured to output a second acoustical signal in response to ambient acoustic waves that are incident on the case, and
wherein processing the acoustical signal comprises generating a measure of a physiological activity in the thorax responsively to a difference between the first and second acoustical signals.

25. The method according to claim 24, and comprising prompting the subject to vocalize one or more predefined sounds, wherein processing the acoustical signal comprises analyzing the acoustical signal received from both the first and second acoustic transducers while the subject vocalizes the one or more predefined sounds.

26. The method according to claim 16, wherein the case contains a pressure sensor, which is configured to sense a force applied between the front surface of the case and the thorax, and wherein the method comprises outputting an instruction to the subject to modify the applied force responsively to the sensed force.

27. A method for sensing, comprising:
acquiring an electrical signal from a body surface of the subject;
acquiring an acoustical signal in response to acoustic waves that are emitted from a thorax of the subject in synchronization with the electrical signal;
processing the acoustical signal in order to extract a seismocardiogram (SCG) of the subject;
processing the electrical signal in order to extract an electrocardiogram (ECG) of the subject;
making a comparison between respective features of the SCG and the ECG; and
outputting data indicative of a medical condition of the subject responsively to the comparison.

28. The method according to claim 27, wherein processing the acoustical signal comprises identifying a periodic feature in the ECG and segmenting the SCG using the identified periodic feature and the synchronization of the acoustical signal with the electrical signal.
